(19) United States
(12) Patent Application Publication
(10) Pub. No.: US 2021/0327571 A1
(43) Pub. Date: Oct. 21, 2021

(54) FACILITATION OF AUGMENTED REALITY FOR WELL-BEING ASSESSMENTS

(71) Applicant: AT&T Intellectual Property I, L.P., Atlanta, GA (US)

(72) Inventors: Brittaney Zellner, Smyrna, GA (US); Sameena Khan, Peachtree Corners, GA (US); Ryan Schaub, Berkeley Lake, GA (US); Barrett Kreiner, Woodstock, GA (US); Ari Craine, Marietta, GA (US); Robert Koch, Peachtree Corners, GA (US)

(21) Appl. No.: 16/850,679

(22) Filed: Apr. 16, 2020

(51) Int. Cl. G16H 40/67; G06T 19/00; G16H 40/40; G16H 50/30; G16H 80/00; G02B 27/00; G02B 27/01

(52) U.S. Cl. CPC G16H 40/67; G06T 19/006; G16H 40/40; G02B 2027/0178; G16H 80/00; G02B 27/0093; G02B 27/017; G16H 50/30

(57) ABSTRACT

A viewer can be presented with an augmented reality (AR) view relative to a person to determine a well-being of the person. The disclosed system can enable the well-being of a person or animal to be predicted and displayed via augmented reality. In addition, the system can ensure privacy when only certain users should be permitted to view the AR-displayed information. For example, bone conduction technology can be utilized to ensure that the correct person can view personal data of the person that is being assessed.

### TECHNICAL FIELD

This disclosure relates generally to facilitating augment reality assessments. For example, this disclosure relates to facilitating augmented reality assessments for the well-being of beings.

### BACKGROUND

Augmented reality (AR) is an interactive experience of a real-world environment where the objects that reside in the real world are enhanced by computer-generated perceptual information, sometimes across multiple sensory modalities, including visual, auditory, haptic, somatosensory and olfactory. An augogram is a computer-generated image that is used to create AR. Augography is the science and practice of making augograms for AR. AR can be defined as a system that fulfills three basic features: a combination of real and virtual worlds, real-time interaction, and accurate 3D registration of virtual and real objects. The overlaid sensory information can be constructive (e.g., additive to the natural environment), or destructive (e.g., masking of the natural environment). This experience is seamlessly interwoven with the physical world such that it is perceived as an immersive aspect of the real environment. In this way, augmented reality alters one's ongoing perception of a real-world environment, whereas virtual reality completely replaces the user's real-world environment with a simulated one. Augmented reality is related to two largely synonymous terms: mixed reality and computer-mediated reality.

The above-described background relating to augmented reality for well-being assessments is merely intended to provide a contextual overview of some current issues, and is not intended to be exhaustive. Other contextual information may become further apparent upon review of the following detailed description.

### BRIEF DESCRIPTION OF THE DRAWINGS

Non-limiting and non-exhaustive embodiments of the subject disclosure are described with reference to the following figures, wherein like reference numerals refer to like parts throughout the various views unless otherwise specified.

FIG. 1 illustrates an example wireless communication system in which a network node device (e.g., network node) and user equipment (UE) can implement various aspects and embodiments of the subject disclosure.

FIG. 2 illustrates an example schematic system block diagram of a system for AR well-being assessments according to one or more embodiments.

FIG. 3 illustrates an example schematic system block diagram of a system for AR well-being assessments comprising a real-time video server according to one or more embodiments.

FIG. 4 illustrates an example schematic system block diagram of a system for AR well-being assessments according to one or more embodiments.

FIG. 5 illustrates an example schematic system block diagram of an AR viewing device view according to one or more embodiments.

FIG. 6 illustrates an example flow diagram for a method for facilitating augmented reality well-being assessments according to one or more embodiments.

FIG. 7 illustrates an example flow diagram for a system for facilitating augmented reality well-being assessments according to one or more embodiments.

FIG. 8 illustrates an example flow diagram for a machine-readable medium for facilitating augmented reality well-being assessments according to one or more embodiments.

FIG. 9 illustrates an example block diagram of an example mobile handset operable to engage in a system architecture that facilitates secure wireless communication according to one or more embodiments described herein.

FIG. 10 illustrates an example block diagram of an example computer operable to engage in a system architecture that facilitates secure wireless communication according to one or more embodiments described herein.

### DETAILED DESCRIPTION

In the following description, numerous specific details are set forth to provide a thorough understanding of various embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment,” or “an embodiment,” means that a particular feature, structure, or characteristic described in connection with the embodiment is included in at least one embodiment. Thus, the appearances of the phrase “in one embodiment,” “in one aspect,” or “in an embodiment,” in various places throughout this specification are not necessarily all referring to the same embodiment. Furthermore, the particular features, structures, or characteristics may be combined in any suitable manner in one or more embodiments.

As utilized herein, terms “component,” “system,” “interface,” and the like are intended to refer to a computer-related entity, hardware, software (e.g., in execution), and/or firmware. For example, a component can be a processor, a process running on a processor, an object, an executable, a program, a storage device, and/or a computer. By way of illustration, an application running on a server and the server can be a component. One or more components can reside within a process, and a component can be localized on one computer and/or distributed between two or more computers.

Further, these components can execute from various machine-readable media having various data structures stored thereon. The components can communicate via local and/or remote processes such as in accordance with a signal having one or more data packets (e.g., data from one component interacting with another component in a local system, distributed system, and/or across a network, e.g., the Internet, a local area network, a wide area network, etc. with other systems via the signal).

As another example, a component can be an apparatus with specific functionality provided by mechanical parts operated by electric or electronic circuitry; the electric or electronic circuitry can be operated by a software application or a firmware application executed by one or more processors; the one or more processors can be internal or external to the apparatus and can execute at least a part of the software or firmware application. As yet another example, a component can be an apparatus that provides specific functionality through electronic components without mechanical parts; the electronic components can include one or more processors therein to execute software and/or firmware that confer(s), at least in part, the functionality of the electronic components. In an aspect, a component can emulate an electronic component via a virtual machine, e.g., within a cloud computing system.

The words “exemplary” and/or “demonstrative” are used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

As used herein, the term “infer” or “inference” refers generally to the process of reasoning about, or inferring states of, the system, environment, user, and/or intent from a set of observations as captured via events and/or data. Captured data and events can include user data, device data, environment data, data from sensors, sensor data, application data, implicit data, explicit data, etc. Inference can be employed to identify a specific context or action, or can generate a probability distribution over states of interest based on a consideration of data and events, for example.

Inference can also refer to techniques employed for composing higher-level events from a set of events and/or data. Such inference results in the construction of new events or actions from a set of observed events and/or stored event data, whether the events are correlated in close temporal proximity, and whether the events and data come from one or several event and data sources. Various classification schemes and/or systems (e.g., support vector machines, neural networks, expert systems, Bayesian belief networks, fuzzy logic, and data fusion engines) can be employed in connection with performing automatic and/or inferred action in connection with the disclosed subject matter.

In addition, the disclosed subject matter can be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, machine-readable device, computer-readable carrier, computer-readable media, or machine-readable media. For example, computer-readable media can include, but are not limited to, a magnetic storage device, e.g., hard disk; floppy disk; magnetic strip(s); an optical disk (e.g., compact disk (CD), a digital video disc (DVD), a Blu-ray Disc™ (BD)); a smart card; a flash memory device (e.g., card, stick, key drive); and/or a virtual device that emulates a storage device and/or any of the above computer-readable media.

As an overview, various embodiments are described herein to facilitate augmented reality well-being assessments. For simplicity of explanation, the methods (or algorithms) are depicted and described as a series of acts. It is to be understood and appreciated that the various embodiments are not limited by the acts illustrated and/or by the order of acts. For example, acts can occur in various orders and/or concurrently, and with other acts not presented or described herein. Furthermore, not all illustrated acts may be required to implement the methods. In addition, the methods could alternatively be represented as a series of interrelated states via a state diagram or events. Additionally, the methods described hereafter are capable of being stored on an article of manufacture (e.g., a machine-readable storage medium) to facilitate transporting and transferring such methodologies to computers. The term article of manufacture, as used herein, is intended to encompass a computer program accessible from any computer-readable device, carrier, or media, including a non-transitory machine-readable storage medium.

It should be noted that although various aspects and embodiments have been described herein in the context of 5G, Universal Mobile Telecommunications System (UMTS), and/or Long Term Evolution (LTE), or other next generation networks, the disclosed aspects are not limited to 5G, a UMTS implementation, and/or an LTE implementation as the techniques can also be applied in 3G, 4G or LTE systems. For example, aspects or features of the disclosed embodiments can be exploited in substantially any wireless communication technology. Such wireless communication technologies can include UMTS, Code Division Multiple Access (CDMA), Wi-Fi, Worldwide Interoperability for Microwave Access (WiMAX), General Packet Radio Service (GPRS), Enhanced GPRS, Third Generation Partnership Project (3GPP), LTE, Third Generation Partnership Project 2 (3GPP2) Ultra Mobile Broadband (UMB), High Speed Packet Access (HSPA), Evolved High Speed Packet Access (HSPA+), High-Speed Downlink Packet Access (HSDPA), High-Speed Uplink Packet Access (HSUPA), Zigbee, or another IEEE 802.12 technology. Additionally, substantially all aspects disclosed herein can be exploited in legacy telecommunication technologies.

Described herein are systems, methods, articles of manufacture, and other embodiments or implementations that can facilitate augmented reality well-being assessments. Facilitating augmented reality well-being assessments can be implemented in connection with any type of device with a connection to the communications network (e.g., a mobile handset, a computer, a handheld device, etc.) any Internet of things (TOT) device (e.g., toaster, coffee maker, blinds, music players, speakers, etc.), and/or any connected vehicles (cars, airplanes, space rockets, and/or other at least partially automated vehicles (e.g., drones)). In some embodiments the non-limiting term user equipment (UE) is used. It can refer to any type of wireless device that communicates with a radio network node in a cellular or mobile communication system. Examples of UE are target device, device to device (D2D) UE, machine type UE or UE capable of machine to machine (M2M) communication, PDA, Tablet, mobile terminals, smart phone, laptop embedded equipped (LEE), laptop mounted equipment (LME), USB dongles etc. Note that the terms element, elements and antenna ports can be interchangeably used but carry the same meaning in this disclosure. The embodiments are applicable to single carrier as well as to multicarrier (MC) or carrier aggregation (CA) operation of the UE. The term carrier aggregation (CA) is also called (e.g. interchangeably called) “multi-carrier system”, “multi-cell operation”, “multi-carrier operation”, “multi-carrier” transmission and/or reception.

In some embodiments the non-limiting term radio network node or simply network node is used. It can refer to any type of network node that serves UE is connected to other network nodes or network elements or any radio node from where UE receives a signal. Examples of radio network nodes are Node B, base station (BS), multi-standard radio (MSR) node such as MSR BS, eNode B, network controller, radio network controller (RNC), base station controller (BSC), relay, donor node controlling relay, base transceiver station (BTS), access point (AP), transmission points, transmission nodes, RRU, RRH, nodes in distributed antenna system (DAS) etc.

Cloud radio access networks (RAN) can enable the implementation of concepts such as software-defined network (SDN) and network function virtualization (NFV) in 5G networks. Certain embodiments of this disclosure can comprise an SDN controller that can control routing of traffic within the network and between the network and traffic destinations. The SDN controller can be merged with the 5G network architecture to enable service deliveries via open application programming interfaces (“APIs”) and move the network core towards an all internet protocol (“IP”), cloud based, and software driven telecommunications network. The SDN controller can work with, or take the place of policy and charging rules function (“PCRF”) network elements so that policies such as quality of service and traffic management and routing can be synchronized and managed end to end.

A user, such as a senior citizen needing additional monitoring, or a user who otherwise has difficulty communicating due to a physical or other disability, can make use of various means to monitor their activity. A representation of this data can be viewed in such a way as to associate it with a visual image of the user. Sensor data can be collected via a sensor data collector app and sent to a sensor data repository. This data can comprise data from sensors such as smart watches, emergency pendants, posture sensors, and clothing motion sensors. For example, the sensor can be a wired sensor, as in a telemedicine application, where the subject is hooked up to a body monitor in a rural medical office. It should be noted that the subject can be a human being, an animal, or any other type of living being. The AR can be viewed by nurse or technician in the room with the patient and, through a video link, also by an expert specialist in a remote city, state, or country. The sensor data analyzer can use known machine learning techniques to identify data anomalies, trends, or critical data levels that are informative. Further, the sensor data analyzer can send instructions to an augmented reality (AR) server, indicating information to display via known AR methods as an overlay if the user is being viewed via an AR viewer.

In order for the sensor data to be associated with the correct user, a digital representation of the user's facial image (e.g., as is used in facial recognition applications) can be used as an index to store and retrieve the sensor data. In this manner, the facial digital representation can be used as a marker reference for AR data presentation. As an example, motion or tactile sensors in the user's clothing can record abrasive motion, which can be an indication of rubbing or scratching. An event recording this can be stored as data in the repository. If the sensor data analyzer determines that there are a significant number of such events, perhaps particularly all at a common location, it can conclude that the user is conducting repetitive scratching or rubbing at a site, perhaps an indication of a rash or pain. Thus, an associated message can be sent from the sensor data analyzer to the AR server. Similarly, other sensors or sources of data can send data for analysis. These can include posture sensors, data from smart watches, data from other medical devices such as pacemakers, blood sugar level monitors, or other worn or implanted devices.

An emergency pendant, worn as a necklace around the neck, can include a gyroscope and collect data representing its position. This data can be sent to the data analyzer and used as a measure of the user's body stability or gait stability, since it can rest at the user's body core. As another type of measure of activity, a user's percentage of sedentary time can be measured using a device such as a smart watch. Also, the user's level of social interactivity can be tracked using such a wearable device by sensing proximity to other people who also wear devices and whose proximity can therefore be detected (e.g., via a near field communication (NFC) connection such as Bluetooth). The sensor data analyzer can also compare data from multiple sources to perform audit-type functions and instruct the AR server to display significant findings.

As an example, a rollator can be used by the user to assist in walking. The rollator can have a motion sensor that can be used to record its usage. Data from the rollator can be compared to data from the user's smart watch to determine usage, for instance, that the user is using their rollator only 20% of the time that they are walking, which can be an indication of a safety concern, particularly if the user lives alone. The AR viewer can also comprise a camera. The camera can be used to record video of the user during a time of wellness assessment. The video can be stored in a historic video content database. Subsequent wellness assessments can also record video and a historic video server can analyze changes over time. A comparison of video captures can be used to identify changes in the size of a mole on the user's face or drooping of one side of their face over time, for example.

A real-time video server can be used to enhance video recorded using the camera and using it to visually determine the user's pulse rate, respiration rate, facial twitches, and other factors, using known techniques. When viewing the user, the facial image can be used as the marker to index the correct AR content (e.g., the content for the specific user). The AR view can communicate with the AR server to obtain pertinent information for display, as delivered by the sensor data analyzer, the historic video server, and the real-time video server. A person using the AR viewer can be presented with a display of pertinent data. Displayed data that contains results that are outside a standard threshold can be displayed in an alerting color. The user can interact with the display, for instance, based on an alert, the viewing party can schedule an appointment in real-time by accessing contact and calendar data for the user via a network.

Similarly, if the AR data display indicates an immediate concern, the person viewing the user can use the speaker and microphone to ask for helpful instructions on how to respond to the data. It should be noted that the microphone can be a digital or a non-digital microphone. For example, if the microphone is digital, it can produce audio data, however, the microphone can be non-digital and produce an audio signal that can be digitized by an analog-to-digital converter to produce the outputs for facilitation of the scenarios outlined in this disclosure. The instructions can be retrieved from a database and displayed visually or can be presented via the viewer microphone. Alternatively, the person using the viewer can be connected to another person via a voice or video connection to assist in treating the user. If a video connection is made, the video of the assistant can be presented via an AR stream by the AR server and the audio of the assistant can be presented via the AR viewer speaker.

A private AR view can be presented only to certain persons using the viewer. The viewer can use standard login techniques to determine if the user has a proper privacy access level to view private information. If the AR viewer is a shared resource, other mechanisms can be used to determine the person's viewing access level. For instance, the AR viewer can make a bone conduction reading of the bones in the head of the viewing person. This measurement can be compared with a known reading for a trusted party, such as a family member, which can be stored in a repository accessible via a network. In this manner, for instance, only family members can be presented certain private AR data, which is not viewable by others. An application of this method can be seeing physiological responses to questions about how the user is being treated in a retirement community. For instance, if they are being treated poorly, their response to questions or people entering the room can indicate a potential problem.

It should also be noted that an artificial intelligence (AI) component can facilitate automating one or more features in accordance with the disclosed aspects. A memory and a processor as well as other components can include functionality with regard to the figures. The disclosed aspects in connection with augmented reality space well-being assessments can employ various AI-based schemes for carrying out various aspects thereof. For example, a process for detecting one or more trigger events, generating an augmented image as a result of the one or more trigger events, and modifying one or more reported measurements, and so forth, can be facilitated with an example automatic classifier system and process. In another example, a process for penalizing one augmented reality-based image while preferring another augmented reality based image can be facilitated with the example automatic classifier system and process.

An example classifier can be a function that maps an input attribute vector, x=(x1, x2, x3, x4, xn), to a confidence that the input belongs to a class, that is, f(x)=confidence(class). Such classification can employ a probabilistic and/or statistical-based analysis (e.g., factoring into the analysis utilities and costs) to prognose or infer an action that can be automatically performed.

A support vector machine (SVM) is an example of a classifier that can be employed. The SVM can operate by finding a hypersurface in the space of possible inputs, which the hypersurface attempts to split the triggering criteria from the non-triggering events. Intuitively, this makes the classification correct for testing data that is near, but not identical to training data. Other directed and undirected model classification approaches include, for example, naïve Bayes, Bayesian networks, decision trees, neural networks, fuzzy logic models, and probabilistic classification models providing different patterns of independence can be employed. Classification as used herein also may be inclusive of statistical regression that is utilized to develop models of priority.

The disclosed aspects can employ classifiers that are explicitly trained (e.g., via a generic training data) as well as implicitly trained (e.g., via observing mobile device usage as it relates to triggering events, observing network frequency/technology, receiving extrinsic information, and so on). For example, SVMs can be configured via a learning or training phase within a classifier constructor and feature selection module. Thus, the classifier(s) can be used to automatically learn and perform a number of functions, including but not limited to modifying an image to be output, modifying one or more reported measurements, and so forth. The criteria can include, but is not limited to, predefined values, frequency attenuation tables or other parameters, service provider preferences and/or policies, and so on.

In one embodiment, described herein is a method comprising receiving, by a server device comprising a processor, sensor data representative of a sensed characteristic of a human being. In response to receiving the sensor data, the method can comprise analyzing, by the server device, the sensor data for a variation in the sensor data, the variation in the sensor data representative of a variation of the sensed characteristic. Additionally, in response to analyzing the sensor data, the method can comprise determining, by the server device, that a threshold value associated with the variation of the sensed characteristic has been satisfied. Furthermore, in response to the threshold value being determined to have been satisfied, the method can comprise generating, by the server device, augmented reality image data representative of an augmented reality image to be sent to an augmented reality viewing device to facilitate displaying the augmented reality image at the augmented reality viewing device.

According to another embodiment, a system can facilitate, receiving sensor data representative of a sensed characteristic of a being. In response to the receiving the sensor data, the system can comprise analyzing the sensor data for variation data representative of a variation of the sensed characteristic from a first sensed characteristic to a second sensed characteristic. In response to the analyzing the sensor data, the system can comprise determining that a condition associated with the second sensed characteristic has been satisfied. Furthermore, in response to the condition being determined to have been satisfied, the system can comprise sending augmented reality image data representative of an augmented reality image to an augmented reality device.

According to yet another embodiment, described herein is a machine-readable storage medium that can perform the operations comprising receiving image data representative of physical characteristic of a living entity. In response to the receiving the image data, the machine-readable medium operations can comprise analyzing the image data for variation data representative of a variation of the physical characteristic from a first physical characteristic to a second physical characteristic. In response to the analyzing the image data, the machine-readable medium operations can comprise determining that a condition associated with the second physical characteristic is satisfied. Additionally, in response to the determining that the condition is satisfied, the machine-readable medium operations can comprise facilitating sending augmented reality image data representative of an augmented reality image to an augmented reality device.

These and other embodiments or implementations are described in more detail below with reference to the drawings.

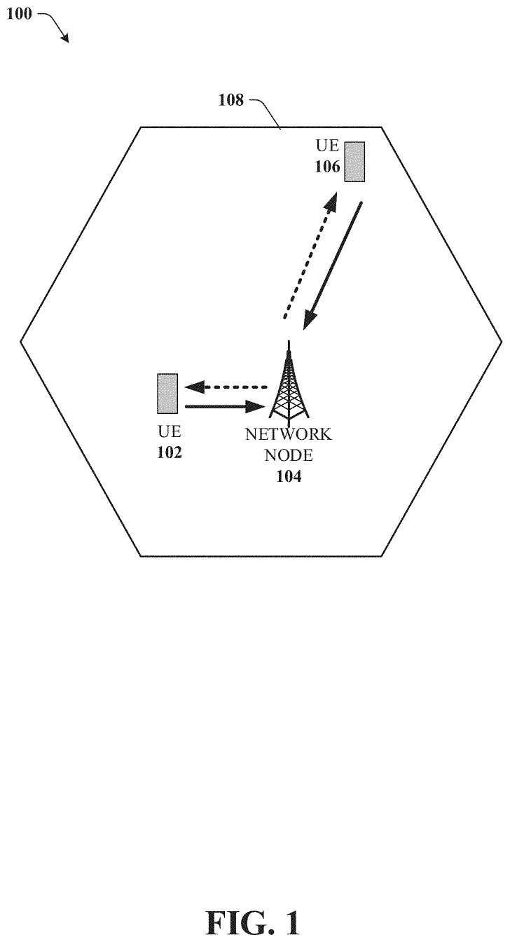

Referring now to FIG. 1, illustrated is an example wireless communication system 100 in accordance with various aspects and embodiments of the subject disclosure. In one or more embodiments, system 100 can comprise one or more user equipment UEs 102. The non-limiting term user equipment can refer to any type of device that can communicate with a network node in a cellular or mobile communication system. A UE can have one or more antenna panels having vertical and horizontal elements. Examples of a UE comprise a target device, device to device (D2D) UE, machine type UE or UE capable of machine to machine (M2M) communications, personal digital assistant (PDA), tablet, mobile terminals, smart phone, laptop mounted equipment (LME), universal serial bus (USB) dongles enabled for mobile communications, a computer having mobile capabilities, a mobile device such as cellular phone, a laptop having laptop embedded equipment (LEE, such as a mobile broadband adapter), a tablet computer having a mobile broadband adapter, a wearable device, a virtual reality (VR) device, a heads-up display (HUD) device, a smart car, a machine-type communication (MTC) device, and the like. User equipment UE 102 can also comprise IOT devices that communicate wirelessly.

In various embodiments, system 100 is or comprises a wireless communication network serviced by one or more wireless communication network providers. In example embodiments, a UE 102 can be communicatively coupled to the wireless communication network via a network node 104. The network node (e.g., network node device) can communicate with user equipment (UE), thus providing connectivity between the UE and the wider cellular network. The UE 102 can send transmission type recommendation data to the network node 104. The transmission type recommendation data can comprise a recommendation to transmit data via a closed loop MIMO mode and/or a rank-1 precoder mode.

A network node can have a cabinet and other protected enclosures, an antenna mast, and multiple antennas for performing various transmission operations (e.g., MIMO operations). Network nodes can serve several cells, also called sectors, depending on the configuration and type of antenna. In example embodiments, the UE 102 can send and/or receive communication data via a wireless link to the network node 104. The dashed arrow lines from the network node 104 to the UE 102 represent downlink (DL) communications and the solid arrow lines from the UE 102 to the network nodes 104 represents an uplink (UL) communication.

System 100 can further include one or more communication service provider networks 106 that facilitate providing wireless communication services to various UEs, including UE 102, via the network node 104 and/or various additional network devices (not shown) included in the one or more communication service provider networks 106. The one or more communication service provider networks 106 can include various types of disparate networks, including but not limited to: cellular networks, femto networks, picocell networks, microcell networks, internet protocol (IP) networks Wi-Fi service networks, broadband service network, enterprise networks, cloud based networks, and the like. For example, in at least one implementation, system 100 can be or include a large scale wireless communication network that spans various geographic areas. According to this implementation, the one or more communication service provider networks 106 can be or include the wireless communication network and/or various additional devices and components of the wireless communication network (e.g., additional network devices and cell, additional UEs, network server devices, etc.). The network node 104 can be connected to the one or more communication service provider networks 106 via one or more backhaul links 108. For example, the one or more backhaul links 108 can comprise wired link components, such as a T1/E1 phone line, a digital subscriber line (DSL) (e.g., either synchronous or asynchronous), an asymmetric DSL (ADSL), an optical fiber backbone, a coaxial cable, and the like. The one or more backhaul links 108 can also include wireless link components, such as but not limited to, line-of-sight (LOS) or non-LOS links which can include terrestrial air-interfaces or deep space links (e.g., satellite communication links for navigation).

Wireless communication system 100 can employ various cellular systems, technologies, and modulation modes to facilitate wireless radio communications between devices (e.g., the UE 102 and the network node 104). While example embodiments might be described for 5G new radio (NR) systems, the embodiments can be applicable to any radio access technology (RAT) or multi-RAT system where the UE operates using multiple carriers e.g. LTE FDD/TDD, GSM/GERAN, CDMA2000 etc.

For example, system 100 can operate in accordance with global system for mobile communications (GSM), universal mobile telecommunications service (UMTS), long term evolution (LTE), LTE frequency division duplexing (LTE FDD, LTE time division duplexing (TDD), high speed packet access (HSPA), code division multiple access (CDMA), wideband CDMA (WCMDA), CDMA2000, time division multiple access (TDMA), frequency division multiple access (FDMA), multi-carrier code division multiple access (MC-CDMA), single-carrier code division multiple access (SC-CDMA), single-carrier FDMA (SC-FDMA), orthogonal frequency division multiplexing (OFDM), discrete Fourier transform spread OFDM (DFT-spread OFDM) single carrier FDMA (SC-FDMA), Filter bank based multi-carrier (FBMC), zero tail DFT-spread-OFDM (ZT DFT-s-OFDM), generalized frequency division multiplexing (GFDM), fixed mobile convergence (FMC), universal fixed mobile convergence (UFMC), unique word OFDM (UW-OFDM), unique word DFT-spread OFDM (UW DFT-Spread-OFDM), cyclic prefix OFDM CP-OFDM, resource-block-filtered OFDM, Wi Fi, WLAN, WiMax, and the like. However, various features and functionalities of system 100 are particularly described wherein the devices (e.g., the UEs 102 and the network device 104) of system 100 are configured to communicate wireless signals using one or more multi carrier modulation schemes, wherein data symbols can be transmitted simultaneously over multiple frequency subcarriers (e.g., OFDM, CP-OFDM, DFT-spread OFMD, UFMC, FMBC, etc.). The embodiments are applicable to single carrier as well as to multicarrier (MC) or carrier aggregation (CA) operation of the UE. The term carrier aggregation (CA) is also called (e.g. interchangeably called) “multi-carrier system”, “multi-cell operation”, “multi-carrier operation”, “multi-carrier” transmission and/or reception. Note that some embodiments are also applicable for Multi RAB (radio bearers) on some carriers (that is data plus speech is simultaneously scheduled).

In various embodiments, system 100 can be configured to provide and employ 5G wireless networking features and functionalities. 5G wireless communication networks are expected to fulfill the demand of exponentially increasing data traffic and to allow people and machines to enjoy gigabit data rates with virtually zero latency. Compared to 4G, 5G supports more diverse traffic scenarios. For example, in addition to the various types of data communication between conventional UEs (e.g., phones, smartphones, tablets, PCs, televisions, Internet enabled televisions, etc.) supported by 4G networks, 5G networks can be employed to support data communication between smart cars in association with driverless car environments, as well as machine type communications (MTCs). Considering the drastic different communication needs of these different traffic scenarios, the ability to dynamically configure waveform parameters based on traffic scenarios while retaining the benefits of multi carrier modulation schemes (e.g., OFDM and related schemes) can provide a significant contribution to the high speed/capacity and low latency demands of 5G networks. With waveforms that split the bandwidth into several sub-bands, different types of services can be accommodated in different sub-bands with the most suitable waveform and numerology, leading to an improved spectrum utilization for 5G networks.

To meet the demand for data centric applications, features of proposed 5G networks may comprise: increased peak bit rate (e.g., 20 Gbps), larger data volume per unit area (e.g., high system spectral efficiency—for example about 3.5 times that of spectral efficiency of long term evolution (LTE) systems), high capacity that allows more device connectivity both concurrently and instantaneously, lower battery/power consumption (which reduces energy and consumption costs), better connectivity regardless of the geographic region in which a user is located, a larger numbers of devices, lower infrastructural development costs, and higher reliability of the communications. Thus, 5G networks may allow for: data rates of several tens of megabits per second should be supported for tens of thousands of users, 1 gigabit per second to be offered simultaneously to tens of workers on the same office floor, for example; several hundreds of thousands of simultaneous connections to be supported for massive sensor deployments; improved coverage, enhanced signaling efficiency; reduced latency compared to LTE.

The upcoming 5G access network may utilize higher frequencies (e.g., >6 GHz) to aid in increasing capacity. Currently, much of the millimeter wave (mmWave) spectrum, the band of spectrum between 30 gigahertz (Ghz) and 300 Ghz is underutilized. The millimeter waves have shorter wavelengths that range from 10 millimeters to 1 millimeter, and these mmWave signals experience severe path loss, penetration loss, and fading. However, the shorter wavelength at mmWave frequencies also allows more antennas to be packed in the same physical dimension, which allows for large-scale spatial multiplexing and highly directional beamforming.

Performance can be improved if both the transmitter and the receiver are equipped with multiple antennas. Multi-antenna techniques can significantly increase the data rates and reliability of a wireless communication system. The use of multiple input multiple output (MIMO) techniques, which was introduced in the third-generation partnership project (3GPP) and has been in use (including with LTE), is a multi-antenna technique that can improve the spectral efficiency of transmissions, thereby significantly boosting the overall data carrying capacity of wireless systems. The use of multiple-input multiple-output (MIMO) techniques can improve mmWave communications, and has been widely recognized a potentially important component for access networks operating in higher frequencies. MIMO can be used for achieving diversity gain, spatial multiplexing gain and beamforming gain. For these reasons, MIMO systems are an important part of the 3rd and 4th generation wireless systems, and are planned for use in 5G systems.

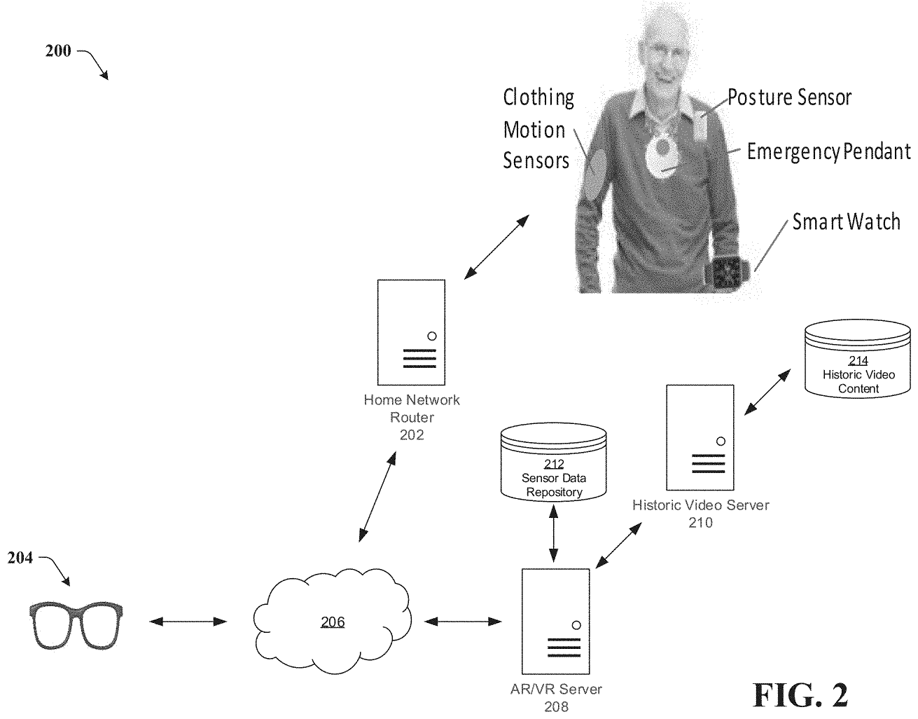

Referring now to FIG. 2, illustrated is an example schematic system block diagram of a system 200 for AR well-being assessments according to one or more embodiments.

Sensor data (e.g. posture data, clothing motion data, emergency data, smart watch data, etc.) can be collected via a sensor data collector app of a mobile device and sent (via a home network router 202) to a sensor data repository 212 via a server device 206 of a cloud-based network. A sensor data analyzer of the sensor data repository 212 can use known machine learning techniques to identify data anomalies, trends, and/or critical data levels that are informative. Further, the sensor data analyzer can send instructions to an augmented reality (AR)/virtual reality (VR) server 208, indicating information to display via known AR methods as an overlay if the user is being viewed via an AR viewer device 204. It should be noted that the sensor data repository 212 can be the sensor data analyzer or the sensor data analyzer can comprise the sensor data repository 212 or vice versa.

If the sensor data analyzer determines that there are a significant number of well-being based events, it can conclude that the subject is conducting repetitive scratching or rubbing at a site, perhaps an indication of a rash or pain. Thus, an associated message can be sent from the sensor data repository 212 to the AR/VR server 208. Similarly, other sensors or sources of data can send data for analysis.

An emergency pendant, worn as a necklace around the neck, can include a gyroscope and collect data representing its position. This data can be sent to the sensor data analyzer and used as a measure of the user's body stability or gait stability, since it can rest at the user's body core. Also, the subjects level of social interactivity can be tracked using such a wearable device by sensing proximity to other people who also wear devices and whose proximity can therefore be detected (e.g., via a near field communication (NFC) connection such as Bluetooth). The sensor data analyzer can also compare data from multiple sources to perform audit-type functions and instruct the AR server device 208 to display significant findings.

The AR viewer device 204 can also comprise a camera. The camera can be used to record video of the subject during a time of wellness assessment. The video can be stored in a historic video content repository 214. Subsequent wellness assessments can also record video and a historic video server 210 can analyze changes over time. For instance, a comparison (via the historic video server 210) of a current video capture to video capture that has been stored in the historic video content repository 214 can be used to identify changes in the size of a mole on the subject's face or drooping of one side of their face over time.

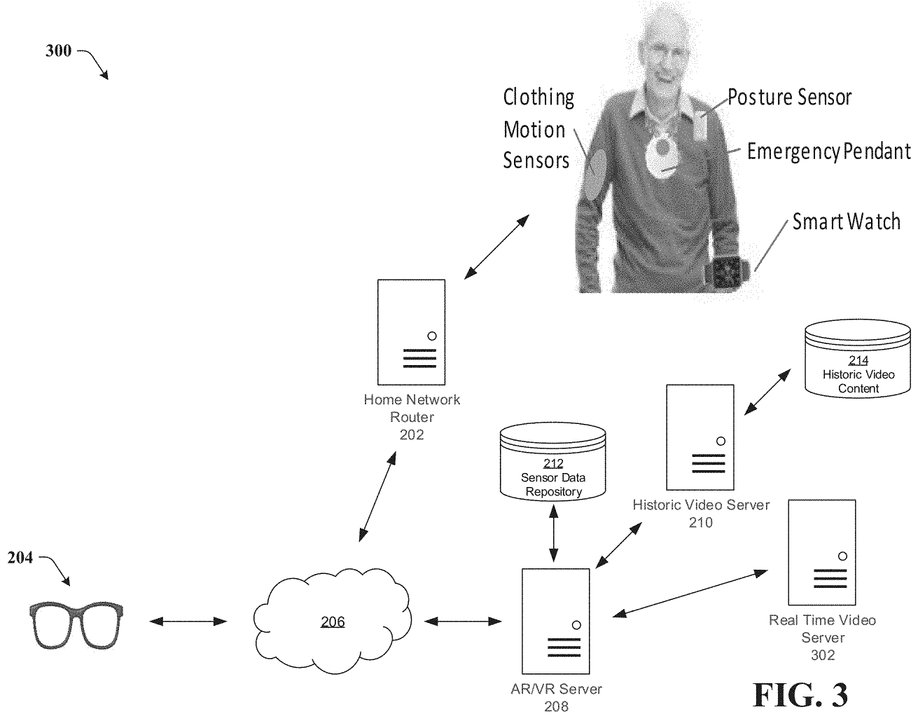

Referring now to FIG. 3, illustrated is an example schematic system block diagram of a system for AR well-being assessments comprising a real-time video server according to one or more embodiments.

A real-time video server 302 can be used to enhance video recorded using the camera of the AR viewer device 204 and using it to visually determine the user's pulse rate, respiration rate, facial twitches, and other factors, using known techniques. When viewing the user, the facial image can be used as the marker to index the correct AR content (e.g., the content for the specific user). The AR viewer device 204 can communicate with the AR/VR server 208 to obtain pertinent information for display, as delivered by the sensor data analyzer, the historic video server 210, and the real-time video server 302. A person using the AR viewer device 204 can be presented with a display of pertinent data. Displayed data that contains results that are outside a standard threshold can be displayed in an alerting color. The user can interact with the display, for instance, based on an alert, the viewing party can schedule an appointment in real-time by accessing contact and calendar data for the user via a network.

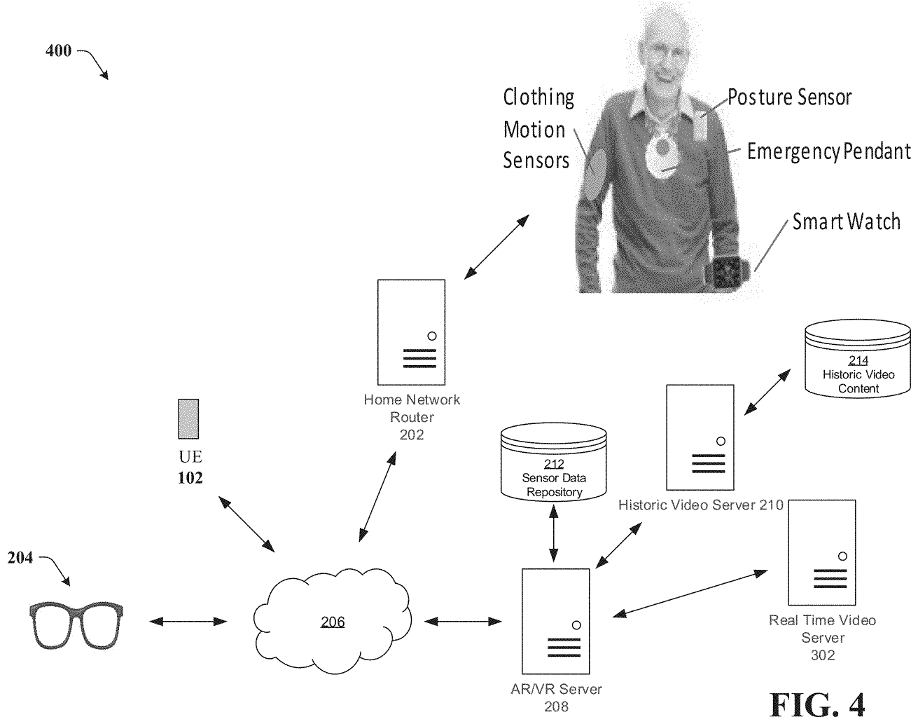

Referring now to FIG. 4, illustrated is an example schematic system block diagram of a system for AR well-being assessments according to one or more embodiments. Similarly, if the AR data display can indicate an immediate concern. A person viewing the subject can use a speaker and/or a microphone of the AR viewer device 204 to ask for helpful instructions on how to respond to the data. The instructions can be retrieved from a database and displayed visually on the AR viewer device 204 or can be presented via the viewer microphone. For example, a family member who is viewing the subject (e.g., an older relative who's been in declining health) via the AR viewer device 204 can determine, based on the AR displayed data, that the subject is exhibiting symptoms that should prompt the family member to call a doctor or other medical professional. Alternatively, the person using the AR viewer device 204 can be connected to the other person (e.g., the doctor or other medical professional), that is utilizing the UE 102, via a voice or video connection to assist in treating the subject. If a video connection is made between the AR viewer device 204 and the UE 102, a video of the other person (e.g., assistant) can be presented via an AR stream by the AR/VR server 208 to the AR viewer device 204, and the audio of the assistant can be presented via the AR viewer device 204 speaker.

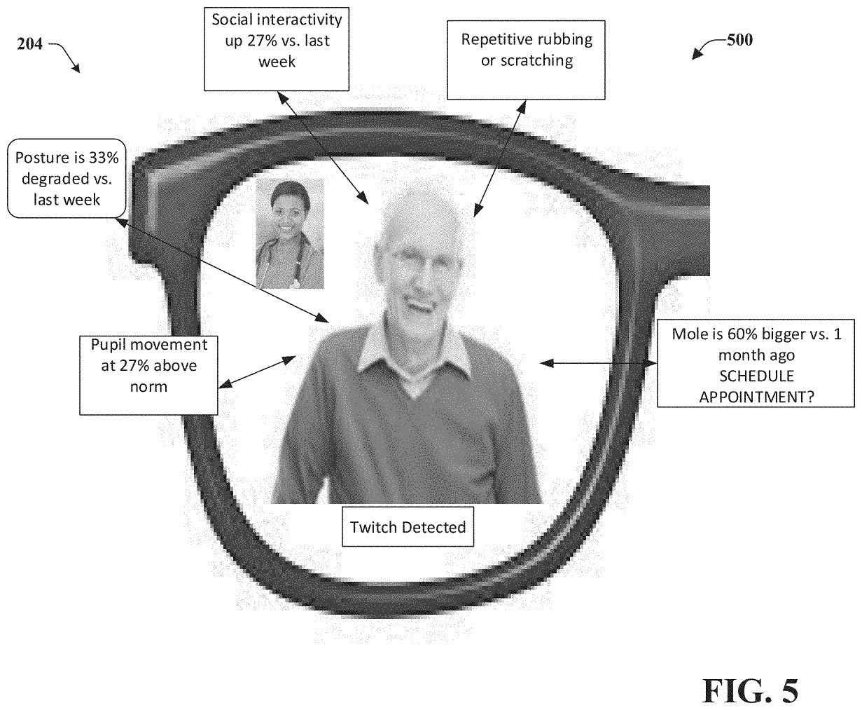

Referring now to FIG. 5 illustrates an example schematic system block diagram of an AR viewing device view 500 according to one or more embodiments.

As mentioned above, the AR view of the AR viewer device 204 can comprise notations (as illustrated) regarding the viewed subject. Additionally, a video of an assistant can be displayed via the AR viewer device 204 view 500. It should be noted that A private AR view can be presented only to certain persons using the AR viewer device 204. The viewer can use standard login techniques to determine a proper privacy access level to view private information. If the AR viewer device 204 is a shared resource, other mechanisms can be used to determine the person's viewing access level. For instance, the AR viewer device 204 can make a bone conduction reading of the bones in the head of the viewing person. This measurement can be compared with a known reading for a trusted party, such as a family member, which can be stored in a repository accessible via the server device 206 of a cloud-based network. In this manner, for instance, only family members can be presented certain private AR data, which is not viewable by others.

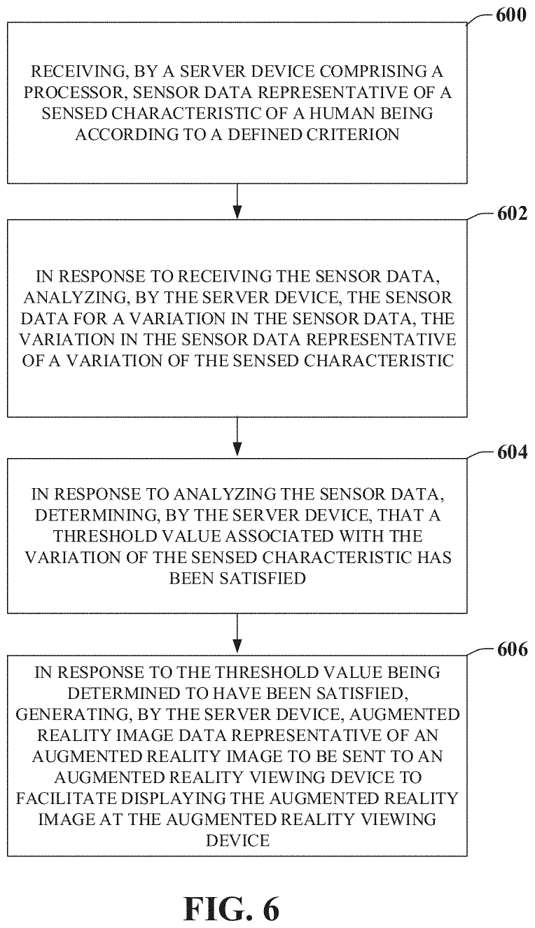

Referring now to FIG. 6, illustrated is an example flow diagram for a method for facilitating augmented reality well-being assessments according to one or more embodiments. At element 600, the method can comprise receiving, by a server device comprising a processor, sensor data representative of a sensed characteristic of a human being. At element 602, in response to receiving the sensor data, the method can comprise analyzing, by the server device, the sensor data for a variation in the sensor data, the variation in the sensor data representative of a variation of the sensed characteristic. Additionally, at element 604, in response to analyzing the sensor data, the method can comprise determining, by the server device, that a threshold value associated with the variation of the sensed characteristic has been satisfied. Furthermore, at element 606, in response to the threshold value being determined to have been satisfied, the method can comprise generating, by the server device, augmented reality image data representative of an augmented reality image to be sent to an augmented reality viewing device to facilitate displaying the augmented reality image at the augmented reality viewing device.

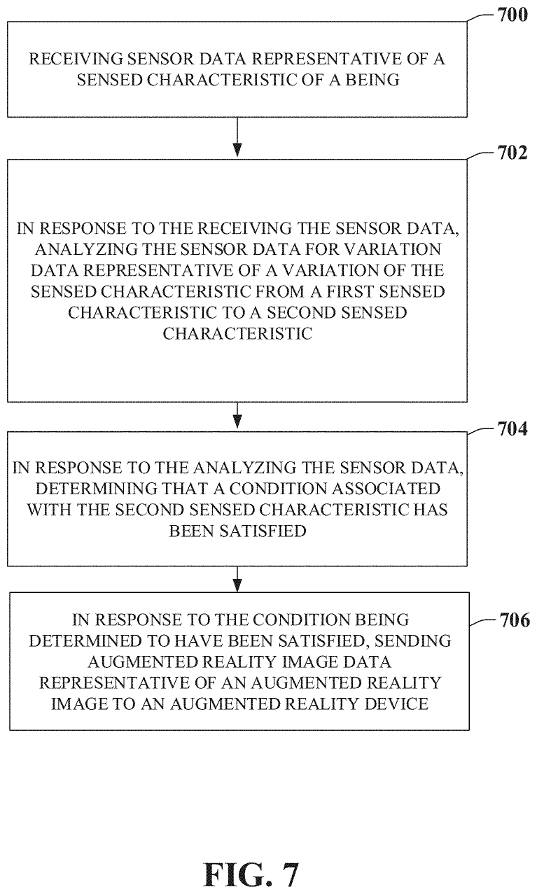

Referring now to FIG. 7, illustrated is an example flow diagram for a system for facilitating augmented reality well-being assessments according to one or more embodiments. At element 700, the system can facilitate, receiving sensor data representative of a sensed characteristic of a being. In response to the receiving the sensor data, at element 702, the system can comprise analyzing the sensor data for variation data representative of a variation of the sensed characteristic from a first sensed characteristic to a second sensed characteristic. In response to the analyzing the sensor data, at element 704, the system can comprise determining that a condition associated with the second sensed characteristic has been satisfied. Furthermore, in response to the condition being determined to have been satisfied, at element 706, the system can comprise sending augmented reality image data representative of an augmented reality image to an augmented reality device.

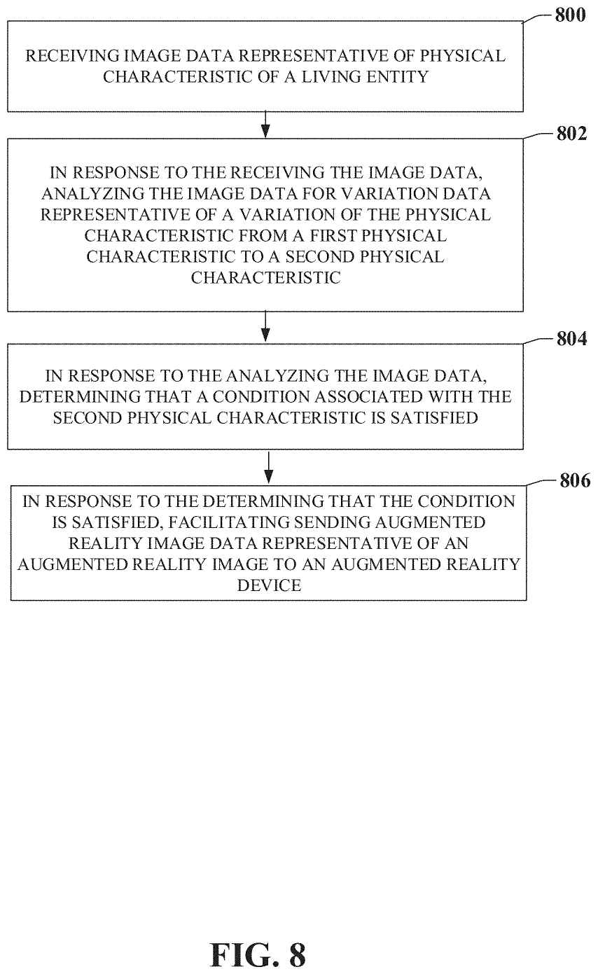

Referring now to FIG. 8, illustrated is an example flow diagram for a machine-readable medium for facilitating augmented reality well-being assessments according to one or more embodiments. At element 800, the machine-readable storage medium can perform the operations comprising receiving image data representative of physical characteristic of a living entity. In response to the receiving the image data, at element 802, the machine-readable medium operations can comprise analyzing the image data for variation data representative of a variation of the physical characteristic from a first physical characteristic to a second physical characteristic. In response to the analyzing the image data, at element 804, the machine-readable medium operations can comprise determining that a condition associated with the second physical characteristic is satisfied. Additionally, at element 806, in response to the determining that the condition is satisfied, the machine-readable medium operations can comprise facilitating sending augmented reality image data representative of an augmented reality image to an augmented reality device.

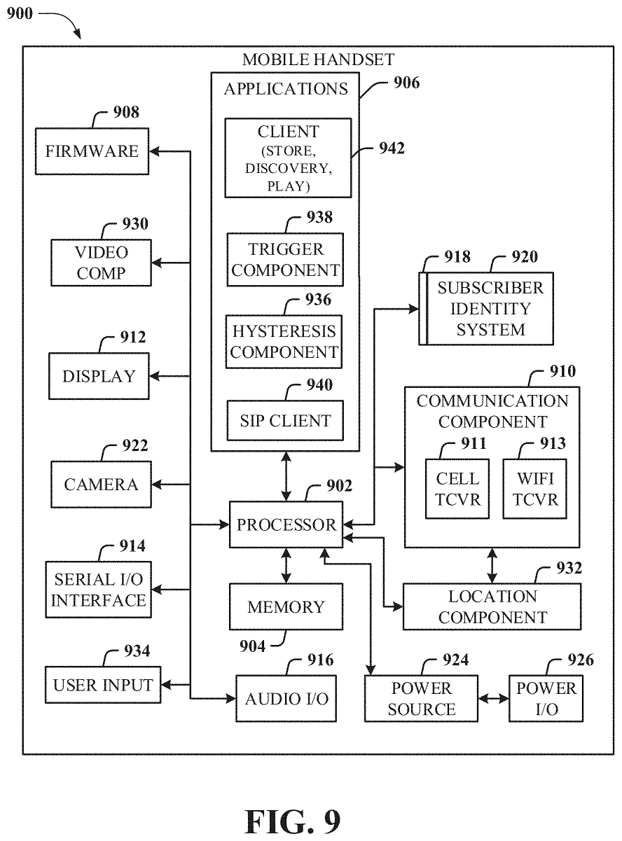

Referring now to FIG. 9, illustrated is a schematic block diagram of an exemplary end-user device such as a mobile device capable of connecting to a network in accordance with some embodiments described herein. Although a mobile handset 900 is illustrated herein, it will be understood that other devices can be a mobile device, and that the mobile handset 900 is merely illustrated to provide context for the embodiments of the various embodiments described herein. The following discussion is intended to provide a brief, general description of an example of a suitable environment 900 in which the various embodiments can be implemented. While the description includes a general context of computer-executable instructions embodied on a machine-readable storage medium, those skilled in the art will recognize that the innovation also can be implemented in combination with other program modules and/or as a combination of hardware and software.

Generally, applications (e.g., program modules) can include routines, programs, components, data structures, etc., that perform particular tasks or implement particular abstract data types. Moreover, those skilled in the art will appreciate that the methods described herein can be practiced with other system configurations, including single-processor or multiprocessor systems, minicomputers, mainframe computers, as well as personal computers, hand-held computing devices, microprocessor-based or programmable consumer electronics, and the like, each of which can be operatively coupled to one or more associated devices.

A computing device can typically include a variety of machine-readable media. Machine-readable media can be any available media that can be accessed by the computer and includes both volatile and non-volatile media, removable and non-removable media. By way of example and not limitation, computer-readable media can comprise computer storage media and communication media. Computer storage media can include volatile and/or non-volatile media, removable and/or non-removable media implemented in any method or technology for storage of information, such as computer-readable instructions, data structures, program modules or other data. Computer storage media can include, but is not limited to, RAM, ROM, EEPROM, flash memory or other memory technology, CD ROM, digital video disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

The handset 900 includes a processor 902 for controlling and processing all onboard operations and functions. A memory 904 interfaces to the processor 902 for storage of data and one or more applications 906 (e.g., a video player software, user feedback component software, etc.). Other applications can include voice recognition of predetermined voice commands that facilitate initiation of the user feedback signals. The applications 906 can be stored in the memory 904 and/or in a firmware 908, and executed by the processor 902 from either or both the memory 904 or/and the firmware 908. The firmware 908 can also store startup code for execution in initializing the handset 900. A communications component 910 interfaces to the processor 902 to facilitate wired/wireless communication with external systems, e.g., cellular networks, VoIP networks, and so on. Here, the communications component 910 can also include a suitable cellular transceiver 911 (e.g., a GSM transceiver) and/or an unlicensed transceiver 913 (e.g., Wi-Fi, WiMax) for corresponding signal communications. The handset 900 can be a device such as a cellular telephone, a PDA with mobile communications capabilities, and messaging-centric devices. The communications component 910 also facilitates communications reception from terrestrial radio networks (e.g., broadcast), digital satellite radio networks, and Internet-based radio services networks.

The handset 900 includes a display 912 for displaying text, images, video, telephony functions (e.g., a Caller ID function), setup functions, and for user input. For example, the display 912 can also be referred to as a “screen” that can accommodate the presentation of multimedia content (e.g., music metadata, messages, wallpaper, graphics, etc.). The display 912 can also display videos and can facilitate the generation, editing and sharing of video quotes. A serial I/O interface 914 is provided in communication with the processor 902 to facilitate wired and/or wireless serial communications (e.g., USB, and/or IEEE 1394) through a hardwire connection, and other serial input devices (e.g., a keyboard, keypad, and mouse). This supports updating and troubleshooting the handset 900, for example. Audio capabilities are provided with an audio I/O component 916, which can include a speaker for the output of audio signals related to, for example, indication that the user pressed the proper key or key combination to initiate the user feedback signal. The audio I/O component 916 also facilitates the input of audio signals through a microphone to record data and/or telephony voice data, and for inputting voice signals for telephone conversations.

The handset 900 can include a slot interface 918 for accommodating a SIC (Subscriber Identity Component) in the form factor of a card Subscriber Identity Module (SIM) or universal SIM 920, and interfacing the SIM card 920 with the processor 902. However, it is to be appreciated that the SIM card 920 can be manufactured into the handset 900, and updated by downloading data and software.

The handset 900 can process IP data traffic through the communication component 910 to accommodate IP traffic from an IP network such as, for example, the Internet, a corporate intranet, a home network, a person area network, etc., through an ISP or broadband cable provider. Thus, VoIP traffic can be utilized by the handset 900 and IP-based multimedia content can be received in either an encoded or decoded format.

A video processing component 922 (e.g., a camera) can be provided for decoding encoded multimedia content. The video processing component 922 can aid in facilitating the generation, editing and sharing of video quotes. The handset 900 also includes a power source 924 in the form of batteries and/or an AC power subsystem, which power source 924 can interface to an external power system or charging equipment (not shown) by a power I/O component 926.

The handset 900 can also include a video component 930 for processing video content received and, for recording and transmitting video content. For example, the video component 930 can facilitate the generation, editing and sharing of video quotes. A location tracking component 932 facilitates geographically locating the handset 900. As described hereinabove, this can occur when the user initiates the feedback signal automatically or manually. A user input component 934 facilitates the user initiating the quality feedback signal. The user input component 934 can also facilitate the generation, editing and sharing of video quotes. The user input component 934 can include such conventional input device technologies such as a keypad, keyboard, mouse, stylus pen, and/or touch screen, for example.

Referring again to the applications 906, a hysteresis component 936 facilitates the analysis and processing of hysteresis data, which is utilized to determine when to associate with the access point. A software trigger component 938 can be provided that facilitates triggering of the hysteresis component 938 when the Wi-Fi transceiver 913 detects the beacon of the access point. A SIP client 940 enables the handset 900 to support SIP protocols and register the subscriber with the SIP registrar server. The applications 906 can also include a client 942 that provides at least the capability of discovery, play and store of multimedia content, for example, music.

The handset 900, as indicated above related to the communications component 910, includes an indoor network radio transceiver 913 (e.g., Wi-Fi transceiver). This function supports the indoor radio link, such as IEEE 802.11, for the dual-mode GSM handset 900. The handset 900 can accommodate at least satellite radio services through a handset that can combine wireless voice and digital radio chipsets into a single handheld device.

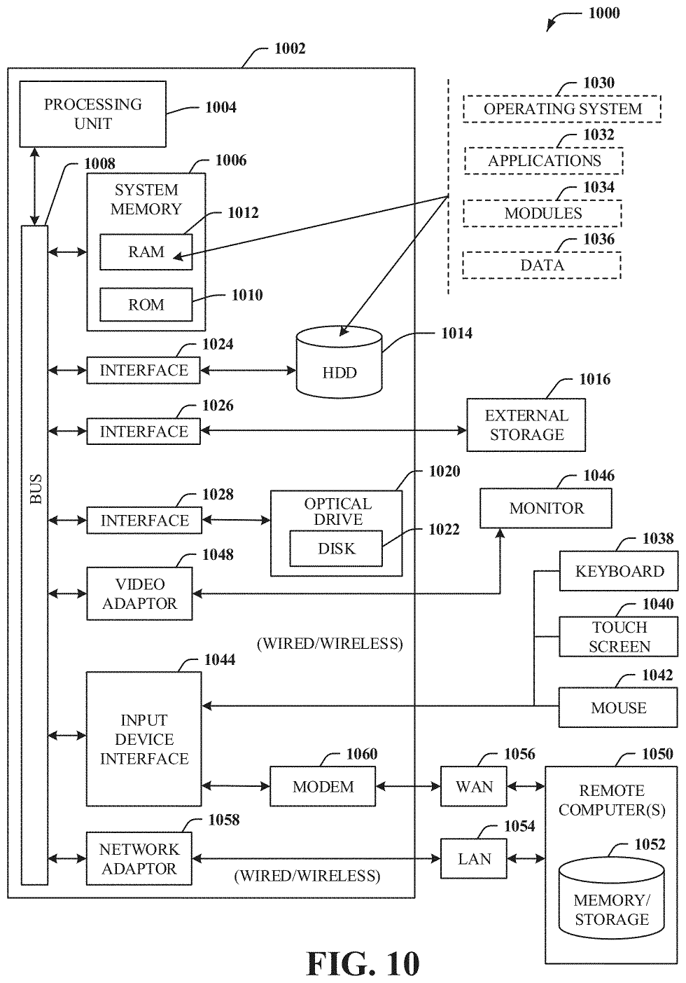

In order to provide additional context for various embodiments described herein, FIG. 10 and the following discussion are intended to provide a brief, general description of a suitable computing environment 1000 in which the various embodiments of the embodiment described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the disclosed methods can be practiced with other computer system configurations, including single-processor or multiprocessor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 10, the example environment 1000 for implementing various embodiments of the aspects described herein includes a computer 1002, the computer 1002 including a processing unit 1004, a system memory 1006 and a system bus 1008. The system bus 1008 couples system components including, but not limited to, the system memory 1006 to the processing unit 1004. The processing unit 1004 can be any of various commercially available processors. Dual microprocessors and other multi-processor architectures can also be employed as the processing unit 1004.

The system bus 1008 can be any of several types of bus structure that can further interconnect to a memory bus (with or without a memory controller), a peripheral bus, and a local bus using any of a variety of commercially available bus architectures. The system memory 1006 includes ROM 1010 and RAM 1012. A basic input/output system (BIOS) can be stored in a non-volatile memory such as ROM, erasable programmable read only memory (EPROM), EEPROM, which BIOS contains the basic routines that help to transfer information between elements within the computer 1002, such as during startup. The RAM 1012 can also include a high-speed RAM such as static RAM for caching data.

The computer 1002 further includes an internal hard disk drive (HDD) 1014 (e.g., EIDE, SATA), one or more external storage devices 1016 (e.g., a magnetic floppy disk drive (FDD) 1016, a memory stick or flash drive reader, a memory card reader, etc.) and an optical disk drive 1020 (e.g., which can read or write from a CD-ROM disc, a DVD, a BD, etc.). While the internal HDD 1014 is illustrated as located within the computer 1002, the internal HDD 1014 can also be configured for external use in a suitable chassis (not shown). Additionally, while not shown in environment 1000, a solid state drive (SSD) could be used in addition to, or in place of, an HDD 1014. The HDD 1014, external storage device(s) 1016 and optical disk drive 1020 can be connected to the system bus 1008 by an HDD interface 1024, an external storage interface 1026 and an optical drive interface 1028, respectively. The interface 1024 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1002, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1012, including an operating system 1030, one or more application programs 1032, other program modules 1034 and program data 1036. All or portions of the operating system, applications, modules, and/or data can also be cached in the RAM 1012. The systems and methods described herein can be implemented utilizing various commercially available operating systems or combinations of operating systems.

Computer 1002 can optionally comprise emulation technologies. For example, a hypervisor (not shown) or other intermediary can emulate a hardware environment for operating system 1030, and the emulated hardware can optionally be different from the hardware illustrated in FIG. 10. In such an embodiment, operating system 1030 can comprise one virtual machine (VM) of multiple VMs hosted at computer 1002. Furthermore, operating system 1030 can provide runtime environments, such as the Java runtime environment or the .NET framework, for applications 1032. Runtime environments are consistent execution environments that allow applications 1032 to run on any operating system that includes the runtime environment. Similarly, operating system 1030 can support containers, and applications 1032 can be in the form of containers, which are lightweight, standalone, executable packages of software that include, e.g., code, runtime, system tools, system libraries and settings for an application.

Further, computer 1002 can be enable with a security module, such as a trusted processing module (TPM). For instance with a TPM, boot components hash next in time boot components, and wait for a match of results to secured values, before loading a next boot component. This process can take place at any layer in the code execution stack of computer 1002, e.g., applied at the application execution level or at the operating system (OS) kernel level, thereby enabling security at any level of code execution.

A user can enter commands and information into the computer 1002 through one or more wired/wireless input devices, e.g., a keyboard 1038, a touch screen 1040, and a pointing device, such as a mouse 1042. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1004 through an input device interface 1044 that can be coupled to the system bus 1008, but can be connected by other interfaces, such as a parallel port, an IEEE 1394 serial port, a game port, a USB port, an IR interface, a BLUETOOTH® interface, etc.

A monitor 1046 or other type of display device can be also connected to the system bus 1008 via an interface, such as a video adapter 1048. In addition to the monitor 1046, a computer typically includes other peripheral output devices (not shown), such as speakers, printers, etc.

The computer 1002 can operate in a networked environment using logical connections via wired and/or wireless communications to one or more remote computers, such as a remote computer(s) 1050. The remote computer(s) 1050 can be a workstation, a server computer, a router, a personal computer, portable computer, microprocessor-based entertainment appliance, a peer device or other common network node, and typically includes many or all of the elements described relative to the computer 1002, although, for purposes of brevity, only a memory/storage device 1052 is illustrated. The logical connections depicted include wired/wireless connectivity to a local area network (LAN) 1054 and/or larger networks, e.g., a wide area network (WAN) 1056. Such LAN and WAN networking environments are commonplace in offices and companies, and facilitate enterprise-wide computer networks, such as intranets, all of which can connect to a global communications network, e.g., the Internet.

When used in a LAN networking environment, the computer 1002 can be connected to the local network 1054 through a wired and/or wireless communication network interface or adapter 1058. The adapter 1058 can facilitate wired or wireless communication to the LAN 1054, which can also include a wireless access point (AP) disposed thereon for communicating with the adapter 1058 in a wireless mode.

When used in a WAN networking environment, the computer 1002 can include a modem 1060 or can be connected to a communications server on the WAN 1056 via other means for establishing communications over the WAN 1056, such as by way of the Internet. The modem 1060, which can be internal or external and a wired or wireless device, can be connected to the system bus 1008 via the input device interface 1044. In a networked environment, program modules depicted relative to the computer 1002 or portions thereof, can be stored in the remote memory/storage device 1052. It will be appreciated that the network connections shown are example and other means of establishing a communications link between the computers can be used.

When used in either a LAN or WAN networking environment, the computer 1002 can access cloud storage systems or other network-based storage systems in addition to, or in place of, external storage devices 1016 as described above. Generally, a connection between the computer 1002 and a cloud storage system can be established over a LAN 1054 or WAN 1056 e.g., by the adapter 1058 or modem 1060, respectively. Upon connecting the computer 1002 to an associated cloud storage system, the external storage interface 1026 can, with the aid of the adapter 1058 and/or modem 1060, manage storage provided by the cloud storage system as it would other types of external storage. For instance, the external storage interface 1026 can be configured to provide access to cloud storage sources as if those sources were physically connected to the computer 1002.

The computer 1002 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

The computer is operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices.

Wi-Fi, or Wireless Fidelity, allows connection to the Internet from a couch at home, a bed in a hotel room, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11 (a, b, g, etc.) to provide secure, reliable, fast wireless connectivity. A Wi-Fi network can be used to connect computers to each other, to the Internet, and to wired networks (which use IEEE 802.3 or Ethernet). Wi-Fi networks operate in the unlicensed 2.4 and 5 GHz radio bands, at an 11 Mbps (802.11a) or 54 Mbps (802.11b) data rate, for example, or with products that contain both bands (dual band), so the networks can provide real-world performance similar to the basic 10BaseT wired Ethernet networks used in many offices.

The above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In this regard, while the subject matter has been described herein in connection with various embodiments and corresponding FIGs, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

What is claimed is:

1. A method, comprising:
receiving, by a server device comprising a processor, sensor data representative of a sensed characteristic of a human being;
in response to receiving the sensor data, analyzing, by the server device, the sensor data for a variation in the sensor data, the variation in the sensor data representative of a variation of the sensed characteristic;
in response to analyzing the sensor data, determining, by the server device, that a threshold value associated with the variation of the sensed characteristic has been satisfied; and
in response to the threshold value being determined to have been satisfied, generating, by the server device, augmented reality image data representative of an augmented reality image to be sent to an augmented reality viewing device to facilitate displaying the augmented reality image at the augmented reality viewing device.

2. The method of claim 1, wherein the sensor data comprises motion data representative of a motion of the being.

3. The method of claim 2, wherein the sensor data further comprises gyroscope data representative of an orientation of the being.

4. The method of claim 1, wherein the augmented reality image to be displayed is a time shifted image representative of a time that is not a current time.

5. The method of claim 1, further comprising:
receiving, by the server device, image data representative of an image of the human being.

6. The method of claim 5, wherein the image data is first image data, wherein the image is a first image, and further comprising:
receiving, by the server device, second image data representative of a second image, the receiving being at a different time than the first image data is received.

7. The method of claim 6, wherein the augmented reality image data is first augmented reality image data, wherein the augmented reality image is a first augmented reality image, and further comprising:
in response to a condition associated with a variance between the first image and the second image being determined to have been satisfied, generating, by the server device, second augmented reality data representative of a second augmented reality image to be sent to the augmented reality viewing device.

8. A system, comprising:
a processor; and
a memory that stores executable instructions that, when executed by the processor, facilitate performance of operations, comprising:
receiving sensor data representative of a sensed characteristic of a living being;
in response to the receiving the sensor data, analyzing the sensor data for variation data representative of a variation of the sensed characteristic from a first sensed characteristic to a second sensed characteristic;
in response to the analyzing the sensor data, determining that a condition associated with the second sensed characteristic has been satisfied; and
in response to the condition being determined to have been satisfied, sending augmented reality image data representative of an augmented reality image to an augmented reality device.

9. The system of claim 8, wherein the augmented reality device comprises eyeglasses equipped with an augmented reality capability.

10. The system of claim 9, wherein the eyeglasses comprise a speaker for output of audio in accordance with the augmented reality image.

11. The system of claim 10, wherein the eyeglasses comprise a microphone for reception of first audio from the living being or second audio generated by a user of the eyeglasses.

12. The system of claim 9, wherein the eyeglasses comprise a camera.

13. The system of claim 12, wherein the operations further comprise:
in response to sending the augmented reality image data, receiving, from the camera, an image of the living being.

14. The system of claim 8, wherein the sensed characteristic comprises a blood sugar level of the living being.

15. A machine-readable medium, comprising executable instructions that, when executed by a processor, facilitate performance of operations, comprising:
receiving image data representative of physical characteristic of a living entity;
in response to the receiving the image data, analyzing the image data for variation data representative of a variation of the physical characteristic from a first physical characteristic to a second physical characteristic;
in response to the analyzing the image data, determining that a condition associated with the second physical characteristic is satisfied; and
in response to the determining that the condition is satisfied, facilitating sending augmented reality image data representative of an augmented reality image to an augmented reality device.

16. The machine-readable medium of claim 15, wherein the second physical characteristic is a facial mole.

17. The machine-readable medium of claim 16, wherein the condition is an increase in size of the facial mole from a first size to a second size that is larger than the first size.

18. The machine-readable medium of claim 17, wherein the augmented reality image comprises threshold data representative of a threshold value that has been satisfied by the second size.

19. The machine-readable medium of claim 18, wherein the operations further comprise:
in response to the threshold data being determined to have been satisfied, facilitating scheduling an appointment for the living entity via the augmented reality device.

20. The machine-readable medium of claim 15, wherein the image data is first image data, and wherein the analyzing comprises comparing the first image data to second image data representative of the second physical characteristic.
